(19) United States
(12) Patent Application Publication
(10) Pub. No.: US 2024/0050595 A1
(43) Pub. Date: Feb. 15, 2024

(54) SHRINKING NANOMATERIAL FOR BIOMEDICAL APPLICATIONS

(71) Applicants: AUBURN UNIVERSITY, Auburn, AL (US); NANOXORT, LLC, Auburn, AL (US)

(72) Inventors: Barry Jay YEH, Aurburn, AL (US); Allan E. DAVID, Aurburn, AL (US); Tareq B. ANANI, Auburn, AL (US); Abhinav SANNIDHI, Aurburn, AL (US)

(21) Appl. No.: 18/232,064

(22) Filed: Aug. 9, 2023

Related U.S. Application Data
(60) Provisional application No. 63/396,676, filed on Aug. 10, 2022.

(51) Int. Cl. A61K 49/18

(52) U.S. Cl. CPC A61K 49/1824

(57) ABSTRACT

A size-changing nanoparticle construct for biomedical applications, and methods to fabricate and use such nanoparticle constructs.

### CROSS-REFERENCE TO RELATED APPLICATIONS

This application claims the benefit under 35 U.S.C. § 119(e) of U.S. Provisional Application Ser. No. 63/396,676, filed on Aug. 10, 2022, the entire disclosure of which is incorporated herein by reference.

### TECHNICAL FIELD

The invention relates to size changing nanoparticle (SCNP) construct for in vivo, in vitro, and/or ex vivo applications.

### BACKGROUND AND SUMMARY OF THE INVENTION

Nanoparticles have been utilized for various applications including but not limited to treatment evaluation, drug delivery, tissue engineering, and in vivo imaging. The conformation of polymer molecules on a nanoparticle depends on several parameters, including solvent properties, charge regulation, and polymer density. Furthermore, it is also understood that polymers grafted to the surface of nanoparticles can be utilized to control size and stability. However, such nanoparticles tend to accumulate in tissues including the liver leading to long-term toxicity concerns. Therefore, it is also important that such nanoparticles can be efficiently removed from circulation. Such an agent could also be amenable for biomedical imaging as it would reduce background noise and allow for repeated administration due to limited tissue accumulation.

Such a nanoparticle construct provides advantages over other agents that remain in the blood as the nanoparticle construct is primarily cleared by renal filtration, avoiding long-term liver deposition and toxicity concerns. Thus, due to an improved ability to compete with other agents, this nanoparticle construct has the potential to achieve widespread clinical adoption.

The following numbered embodiments are contemplated and are non-limiting:
1. An agent comprising a nanoparticle core, and a polymer attached to a surface of the nanoparticle core.
2. The agent of clause 1, any other suitable clause, or any combination of suitable clauses, wherein the agent is configured to change in size upon exposure to a body tissue of a subject.
3. The agent of clause 1, any other suitable clause, or any combination of suitable clauses, wherein the polymer is a first polymer, and wherein the agent further comprises a second polymer.
4. The agent of clause 1, any other suitable clause, or any combination of suitable clauses, wherein the polymer is a polyether, such as polyethylene glycol (PEG), a synthetic or natural polymer, a biopolymers such as a polysaccharide, a protein, a peptides, a DNA, a RNA, or a polyester, a polyanhydride, a polyketone, a vinyl polymer, or any associated co-polymer.
5. The agent of clause 1, any other suitable clause, or any combination of suitable clauses, wherein the agent is stored in a storage solution with a first temperature before being administered into the body tissue with a second temperature, and wherein the polymer is configured to change in size after administration if the first temperature is different from the second temperature.
6. The agent of clause 1, any other suitable clause, or any combination of suitable clauses, wherein the agent is stored in a storage solution with a first ionic strength before being administered into the body tissue with a second ionic strength, and wherein the polymer is configured to change in size after administration if the first ionic strength is different from the second ionic strength.
7. The agent of clause 1, any other suitable clause, or any combination of suitable clauses, wherein the agent is stored in a storage solution with a first pH before being administered into the body tissue with a second pH, and wherein the polymer is configured to change in size after administration if the first pH is different from the second pH.
8. The agent of clause 1, any other suitable clause, or any combination of suitable clauses, wherein the polymer is configured to change in size at a rate determined by pH, ionic strength, or temperature of the body tissue of the subject.
9. The agent of clause 1, any other suitable clause, or any combination of suitable clauses, wherein the polymer is configured to undergo hydrolysis in the body tissue of the subject.
10. The agent of clause 1, any other suitable clause, or any combination of suitable clauses, wherein the change in size of the agent is dependent on a threshold density of the polymer.
11. The agent of clause 1, any other suitable clause, or any combination of suitable clauses, wherein the body tissue is blood.
12. The agent of clause 1, any other suitable clause, or any combination of suitable clauses, wherein the body tissue is a tumor microenvironment.
13. The agent of clause 1, any other suitable clause, or any combination of suitable clauses, wherein the polymer is configured to target a biomarker in the subject.
14. The agent of clause 1, any other suitable clause, or any combination of suitable clauses, wherein, a threshold density of the polymer is tethered to the nanoparticle core.
15. The agent of clause 14, any other suitable clause, or any combination of suitable clauses, wherein the threshold density of the polymer is one half of a theoretical maximum density of the polymer on the nanoparticle core.
16. The agent of clause 14, any other suitable clause, or any combination of suitable clauses, wherein the threshold density of the polymer is one third of a theoretical maximum density of the polymer on the nanoparticle core.
17. The agent of clause 14, any other suitable clause, or any combination of suitable clauses, wherein the threshold density of the polymer is one quarter, of a theoretical maximum density of the polymer on the nanoparticle core.
18. The agent of clause 14, any other suitable clause, or any combination of suitable clauses, wherein the threshold density of the polymer one fifth of a theoretical maximum density of the polymer on the nanoparticle core.
19. The agent of clause 14, any other suitable clause, or any combination of suitable clauses, wherein the threshold density of the polymer one tenth or less of a theoretical maximum density of the polymer on the nanoparticle core.
20. The agent of clause 1, any other suitable clause, or any combination of suitable clauses, wherein agent of claim 1, wherein the nanoparticle core comprises magnetic material.
21. The agent of clause 14, any other suitable clause, or any combination of suitable clauses, wherein the agent comprises at least one reactive site on the surface of the nanoparticle core and at least one reactive polymer tethered to the nanoparticle core.
22. The agent of clause 21, any other suitable clause, or any combination of suitable clauses, wherein a ratio of a number of reactive sites on the agent to a number of reactive polymers is about 1:2.
23. The agent of clause 21, any other suitable clause, or any combination of suitable clauses, wherein a ratio of a number of reactive sites on the agent to a number of reactive polymers is about 1:3.
24. The agent of clause 21, any other suitable clause, or any combination of suitable clauses, wherein a ratio of a number of reactive sites on the agent to a number of reactive polymers is about 1:5, or less.
25. The agent of clause 1, any other suitable clause, or any combination of suitable clauses, wherein the agent can remain in circulation in blood vessels for at least about 5 minutes after administration to the subject.
26. The agent of clause 1, any other suitable clause, or any combination of suitable clauses, wherein the nanoparticle core comprises a coating selected from polyacrylic acid, polyacrylic acid and citric acid, polyacrylic acid-poly(acrylic-co-maleic acid), polyacrylic acid-poly(ethylene glycol) block copolymer, and any combination thereof.
27. The agent of clause 1, any other suitable clause, or any combination of suitable clauses, wherein the agent further comprises a cyclic oligosaccharide with hydrophilic exterior and hydrophobic cavity.
28. The agent of clause 27, any other suitable clause, or any combination of suitable clauses, wherein the cyclic oligosaccharide is β-Cyclodextrin.
29. The agent of clause 1, any other suitable clause, or any combination of suitable clauses, wherein agent is configured to change in size from above about 10 nm to below about 8 nm upon exposure to the body tissue.
30. The agent of clause 1, any other suitable clause, or any combination of suitable clauses, wherein agent is an imaging agent.
31. A method of imaging comprising creating a size-changing imaging agent, administering the size-changing imaging agent into a subject, measuring a signal from the size-changing imaging agent, and removing the size-changing imaging agent from the subject after the size-changing imaging agent changes in size.
32. The method of clause 31, any other suitable clause, or any combination of suitable clauses, wherein the size-changing imaging agent decreases in size based on temperature of a body tissue of the subject.
33. The method of clause 31, any other suitable clause, or any combination of suitable clauses, wherein the size-changing imaging agent decreases in size based on ionic strength of a body tissue of the subject.
34. The method of clause 31, any other suitable clause, or any combination of suitable clauses, wherein the size-changing imaging agent decreases in size based on pH of a body tissue of the subject.
35. The method of clause 31, any other suitable clause, or any combination of suitable clauses, wherein removing the size-changing imaging agent from the subject comprises removal by renal clearance.
36. The method of clause 31, any other suitable clause, or any combination of suitable clauses, wherein the size-changing imaging agent comprises a nanoparticle core and a polymer tethered to the nanoparticle core, and wherein the polymer is configured to change in size upon exposure to a body tissue of the subject.
37. The method of clause 36, any other suitable clause, or any combination of suitable clauses, wherein the polymer is a first polymer, and wherein the imaging agent further comprises a second polymer.
38. The method of clause 36, any other suitable clause, or any combination of suitable clauses, wherein the polymer is PEG.
39. The method of clause 36, any other suitable clause, or any combination of suitable clauses, wherein the method further comprising storing the imaging agent in a storage solution with a first temperature before being administering the imaging agent into the body tissue with a second temperature, and wherein the polymer is configured to change in size after administration if the first temperature is different from the second temperature.
40. The method of clause 36, any other suitable clause, or any combination of suitable clauses, wherein the method storing the imaging agent in a storage solution with a first ionic strength before administering the imaging agent into the body tissue with a second ionic strength, and wherein the polymer is configured to change in size after administration if the first ionic strength is different from the second ionic strength.
41. The method of clause 36, any other suitable clause, or any combination of suitable clauses, wherein the method storing the imaging agent in a storage solution with a first pH before being administering the imaging agent into the body tissue with a second pH, and wherein the polymer is configured to change in size after administration if the first pH is different from the second pH.
42. The method of clause 36, any other suitable clause, or any combination of suitable clauses, wherein the method further comprising storing the imaging agent in a storage solution including an additive with a first temperature before being administering the imaging agent into the body tissue with a second temperature, and wherein the polymer is configured to change in size after administration causes dilution of the additive.
43. The method of clause 36, any other suitable clause, or any combination of suitable clauses, wherein the method storing the imaging agent in a storage solution including an additive with a first ionic strength before administering the imaging agent into the body tissue with a second ionic strength, and wherein the polymer is configured to change in size after administration causes dilution of the additive.
44. The method of clause 36, any other suitable clause, or any combination of suitable clauses, wherein the method storing the imaging agent in a storage solution including an additive with a first pH before being administering the imaging agent into the body tissue with a second pH, and wherein the polymer is configured to change in size after administration causes dilution of the additive.
45. The method of clause 36, any other suitable clause, or any combination of suitable clauses, wherein the polymer is configured to change in size at a rate determined by pH, ionic strength, or temperature of the body tissue of the subject.
46. The method of clause 36, any other suitable clause, or any combination of suitable clauses, wherein the polymer is configured to undergo hydrolysis in the body tissue of the subject.
47. The method of clause 36, any other suitable clause, or any combination of suitable clauses, wherein the change in size of the imaging agent is dependent on a threshold density of the polymer.
48. The method of clause 36, any other suitable clause, or any combination of suitable clauses, wherein the body tissue is blood.
49. The method of clause 36, any other suitable clause, or any combination of suitable clauses, wherein the body tissue is a tumor microenvironment.
50. The method of clause 36, any other suitable clause, or any combination of suitable clauses, wherein the polymer is configured to target a biomarker in the subject.
51. The method of clause 36, any other suitable clause, or any combination of suitable clauses, wherein a threshold density of the polymer is tethered to the nanoparticle core.
52. The method of clause 48, any other suitable clause, or any combination of suitable clauses, wherein the threshold density of the polymer is one half of a theoretical maximum density of the polymer on the nanoparticle core.
53. The method of clause 48, any other suitable clause, or any combination of suitable clauses, wherein the threshold density of the polymer is one third of a theoretical maximum density of the polymer on the nanoparticle core.
54. The method of clause 48, any other suitable clause, or any combination of suitable clauses, wherein the threshold density of the polymer is one quarter of a theoretical maximum density of the polymer on the nanoparticle core.
55. The method of clause 48, any other suitable clause, or any combination of suitable clauses, wherein the threshold density of the polymer is one fifth of a theoretical maximum density of the polymer on the nanoparticle core.
56. The method of clause 48, any other suitable clause, or any combination of suitable clauses, wherein the threshold density of the polymer is one tenth or less of a theoretical maximum density of the polymer on the nanoparticle core.
57. The method of clause 36, any other suitable clause, or any combination of suitable clauses, wherein the nanoparticle core comprises iron.
58. The method of clause 36, any other suitable clause, or any combination of suitable clauses, wherein the imaging agent comprises at least one reactive site on a surface of the nanoparticle core and at least one reactive polymer tethered to the nanoparticle core.
59. The method of clause 58, any other suitable clause, or any combination of suitable clauses, wherein a ratio of a number of reactive sites on the imaging agent to a number of reactive polymers is about 1:2.
60. The method of clause 58, any other suitable clause, or any combination of suitable clauses, wherein a ratio of a number of reactive sites on the imaging agent to a number of reactive polymers is about 1:3.
61. The method of clause 585, any other suitable clause, or any combination of suitable clauses, wherein a ratio of a number of reactive sites on the imaging agent to a number of reactive polymers is about 1:5, or less.
62. The method of clause 31, any other suitable clause, or any combination of suitable clauses, wherein measuring the signal from the size-changing imaging agent comprises measuring the signal for at least about 5 minutes after administering the imaging agent into the subject.
63. A method of fabricating a size-changing agent, comprising synthesizing a nanoparticle core by co-precipitation, stabilizing the nanoparticle core by using a surface coating, and grafting a polymer to the nanoparticle core.
64. The method of clause 63, any other suitable clause, or any combination of suitable clauses, wherein the polymer is PEG and grafting the polymer to the nanoparticle comprises the step of PEGylation.
65. The method of clause 63, any other suitable clause, or any combination of suitable clauses, wherein the nanoparticle core comprises superparamagnetic iron oxide.
66. The method of clause 63, any other suitable clause, or any combination of suitable clauses, wherein the nanoparticle core comprises iron.
67. The method of clause 66, any other suitable clause, or any combination of suitable clauses, wherein the PEG to iron ratio is in the range from about 1.5 to about 5.
68. The method of clause 64, any other suitable clause, or any combination of suitable clauses, wherein the molecular weight of PEG is about 5 kDa.
69. The method of clause 64, any other suitable clause, or any combination of suitable clauses, wherein the molecular weight of PEG is about 2 kDa.
70. The method of clause 63, any other suitable clause, or any combination of suitable clauses, wherein the surface coating is poly(acrylic acid).
71. The method of clause 63, any other suitable clause, or any combination of suitable clauses, wherein the surface coating is polyacrylic acid and citric acid.
72. The method of clause 63, any other suitable clause, or any combination of suitable clauses, wherein the surface coating is polyacrylic acid and polyacrylic-co-maleic acid.
73. The method of clause 63, any other suitable clause, or any combination of suitable clauses, wherein the size-changing agent comprises a diameter of about 8 nm to 30 nm.

### BRIEF DESCRIPTIONS OF THE DRAWINGS

The detailed description particularly refers to the accompanying figures:

FIG. 1 is an embodiment of a nanoparticle construct with a nanoparticle core and a polymer grafted to the surface of the nanoparticle core;

FIG. 2A is a graph illustrating the size of nanoparticle constructs comprising PEG polymers with different densities in solutions with different ionic strengths;

FIG. 2B is a graph illustrating the change in the size of nanoparticle constructs comprising PEG polymers with different densities when transferred to solutions with different ionic strengths;

FIG. 3 is a graph illustrating the rate of change of the size of nanoparticle constructs comprising PEG polymers with different densities when transferred to a 1× PBS solution from water;

FIG. 4A is a representative transmission electron microscopy (TEM) image of PAA-SPIONs showing spherical iron oxide cores with diameters of 4.1 nm±0.9 nm;

FIG. 4B is a graph illustrating size-change studies showing that PEGylated PAA-SPIONs shrink from a hydrodynamic diameter of 18 nm to 13 nm within 15 minutes, as observed by the narrowing of size distribution curves in dynamic light scattering studies;

FIG. 4C is a graph of PAA-SPIONs modified with κ kDa polyethylene glycol (PEG) showing that shrinkage kinetics was dependent on the density of PEG on the surface;

FIG. 4D is a graph of PAA-SPIONs modified with 5 kDa polyethylene glycol (PEG) showing that nanoparticle constructs with high PEG surface coverage did not shrink in size and that nanoparticle constructs with PEG-to-Fe ratio <1 showed size shrinkage with a decrease in size of about 7 nm for the low surface density;

FIG. 5A is a graph illustrating the size of the nanoparticle constructs with polyacrylic acid (PAA), polyacrylic acid and citric acid (PAA+CA), and polyacrylic acid and poly(acrylic-co-maleic acid) (PAA-MA) coating when moved from water to 1× PBS over a period of time;

FIG. 5B is a graph illustrating change in size of the nanoparticle constructs with polyacrylic acid (PAA), polyacrylic acid and citric acid (PAA+CA), and polyacrylic acid and poly(acrylic-co-maleic acid) (PAA-MA) coating when moved from water to 1× PBS over a period of time;

FIG. 6 is a graph illustrating the size of nanoparticle constructs with varying ratios of reactive PEG to the number of amine (NH₂) sites on the nanoparticle construct surface in solutions with different ionic strengths.

FIG. 7A is a graph illustrating the size of the nanoparticle constructs with superparamagnetic iron oxide nanoparticle core (SPIONs) stabilized at varying molar ratios of polyacrylic acid (PAA) and polyacrylic acid-poly(ethylene glycol) block copolymer (PAA-PEG) when moved from water to 1× PBS over a period of time;

FIG. 7B is a graph illustrating the change in size of the nanoparticle constructs with superparamagnetic iron oxide nanoparticle core (SPIONs) stabilized at varying molar ratios of polyacrylic acid (PAA) and polyacrylic acid-poly(ethylene glycol) block copolymer (PAA-PEG) when moved from water to 1× PBS over a period of time

FIG. 8 is a graph illustrating the rate of change of the size of nanoparticle constructs comprising free second PEG when transferred to a 1× PBS solution from water;

FIG. 9A is an illustration of a change in the size of a nanoparticle construct with a medium density of PEG with the incorporation of β-Cyclodextrin;

FIG. 9B is an illustration of a change in the size of a nanoparticle construct with a high density of PEG with the incorporation of β-Cyclodextrin;

FIG. 10A is a graph illustrating the hemolytic effect of different formulations at different concentrations after 2 hour incubation with RBCs from Sprague-Dawley (SD) rats;

FIG. 10B shows the uptake on nanoparticle constructs by Raw 264.7 macrophage cells.

FIG. 10C is a graph illustrating the MTT assay showing cytotoxicity of 2k-SCNP, 5k-SCNP, and Feraheme® at different concentrations;

FIG. 10D is a graph illustrating the SRB assay showing cytotoxicity of 2k-SCNP, 5k-SCNP, and Feraheme® at different concentrations;

FIG. 10E is a graph illustrating the ROS assay showing oxidative stress of 2k-SCNP, 5k-SCNP, and Feraheme® at different concentrations;

FIG. 11A is a graph illustrating plasma concentration vs time curve when fitted with first order kinetics for a pharmacokinetics study in rats injected with 2k-SCNP, 5k-SCNP and Feraheme®;

FIG. 11B is a graph illustrating the concentration decay in the first 2 hours post-injection for a pharmacokinetics study in rats injected with 2k-SCNP, 5k-SCNP and Feraheme®;

FIG. 12A is a graph illustrating a biodistribution study (at 24 hr) showing accumulation of 2k-SCNP, 5k-SCNP, and Feraheme® in liver, spleen, brain, lungs and heart at 24 hours post-injection;

FIG. 12B is a graph illustrating a biodistribution study (at 24 hr) showing accumulation of 2k-SCNP, 5k-SCNP, and Feraheme® in kidneys at 24 hours post-injection;

FIG. 12C is a graph illustrating a biodistribution study (at 24 hr) showing accumulation of 2k-SCNP, 5k-SCNP, and Feraheme® in urine at 24 hours post-injection;

FIG. 12D includes histological images (H&E stained) showing no signs of toxicity in the six tested tissues (brain, kidneys, spleen, lung, liver, and heart);

FIG. 13A illustrates a MRA study with different doses of 2k-SCNP, showing pre-contrast and post-contrast images acquired following an initial dose of 0.01 mmol/kg, and a second higher dose of 0.06 mmol/kg (34 min after 1st injection) of 2k-SCNP;

FIG. 13B illustrates a MRA study with different doses of 2k-SCNP, showing regions of interest (ROIs) around the right common carotid (ROI 1), right subclavian (ROI 2), brachiocephalic (ROI 3), and left common carotid (ROI 4) arteries;

FIG. 13C illustrates a MRA study with different doses of 2k-SCNP, showing a graph illustrating signal-to-noise ratio (SNR) change (%), defined as SNR at time, “t”, relative to SNR at pre-contrast level calculated at different time points for the four vascular ROIs;

FIG. 14A shows pre-contrasts and post-contrasts MRA images after administration of 2k-SCNP, Gadavist, and Feraheme®; and

FIG. 14B illustrates MRA analysis (SNR and CNR change (%)) indicating that 2k-SCNP has an extended imaging window (>100% SNR and CNR change (%)) for four ROIs around right and left common carotid, subclavian, and brachiocephalic arteries.

### DETAILED DESCRIPTION

The present disclosure is directed to a size changing nanoparticle construct. The nanoparticle construct can be utilized as a diagnostic tool, as a therapeutic, as a drug delivery agent, or for cell sorting, filtration or other relevant applications. The present disclosure relates to an iron-based nanoparticle construct that can initially remain at a high concentration in blood vessels (for example, at a concentration similar to the concentration at which the nanoparticle construct has been administered) after administration by avoiding tissue distribution, and then shrink in size for renal clearance. The present disclosure relates to a size-changing nanoparticle construct (SCNP) that allows for improved imaging performance and/or tracking.

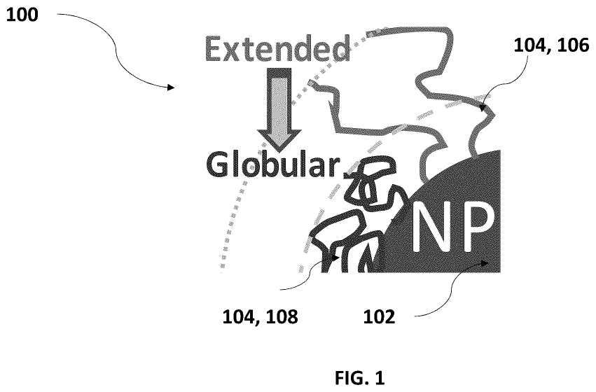

In one aspect, as shown in FIG. 1, the disclosure is directed to a nanoparticle construct 100 that can be visualized by imaging after administration into a subject. The nanoparticle construct 100 can remain in circulation in blood vessels for at least about 5 minutes. In some embodiments, the nanoparticle construct 100 can remain in circulation in blood vessels for at least about 5 minutes to about 10 minutes, from about 10 minutes to about 15 minutes, or from about 15 minutes to about 20 minutes, including any time or range of time comprised therein.

Such prolonged circulation allows for high-quality imaging of the nanoparticle construct. The nanoparticle construct 100 can serve as a blood pool contrast agent (BPCA). In some embodiments, the nanoparticle construct 100 can be a size-changing nanoparticle construct (SCNP) 100 that can change in size depending on the surrounding environment. In some embodiments, this rate of change in the size of the nanoparticle construct 100 allows for the nanoparticle construct 100 to remain at or near maximum concentration in blood for at least 15 minutes following the administration of the nanoparticle construct 100.

The nanoparticle construct 100 includes a nanoparticle core 102 and one or more polymers 104 adsorbed, attached, tethered, or grafted on a surface of the nanoparticle core 102. The conformation of the polymer 104 on the nanoparticle core 102 can affect the efficacy and imaging of the nanoparticle construct 100. The nanoparticle construct 100 can allow for improved magnetic resonance angiography (MRA) imaging while remaining in circulation for a short period of time before shrinking in size from an extended structure 106 to become a globular structure 108 due to the change in configuration, and/or shape of the polymer 104 (see FIG. 1). In some embodiments, the diameter of the nanoparticle construct 100 can be at least about 10 nm and allows for magnetic resonance angiography imaging. In other embodiments, the diameter of the nanoparticle construct 100 can range from about 8 nm to about 10 nm, about 10 nm to about 12 nm, or about 12 nm to about 16 nm, including any size or range comprised therein.

The polymer 104 can be PEG, polyacrylamide (PAA), poly(methyl acrylate (PMA), poly (methyl methacrylate (PMMA), poly(ter butyl acrylate), polycaprolactone, or polystyrene. In some embodiments, the polymer 104 can be a synthetic or natural polymer, a biopolymers such as a polysaccharide, a protein, a peptides, a DNA, a RNA, or a polyesters, a polyanhydrides, a polyketones, a vinyl polymers, or any associated co-polymer. The nanoparticle core 102 can be iron (Fe), superparamagnetic iron oxide (SPIONs), other magnetic core (e.g. zinc, manganese, or gadolinium), or nonmagnetic core (e.g. gold, silica). In some embodiments, the polymer 104 is about 8 nm to about 30 nm and can be PEG, and the PEG can be attached to the nanoparticle core 102 by PEGylation. In some embodiments, the PEG-to-Fe ratio in the reaction can range from about 0.15 to about 5, including any ratio or range comprised therein.

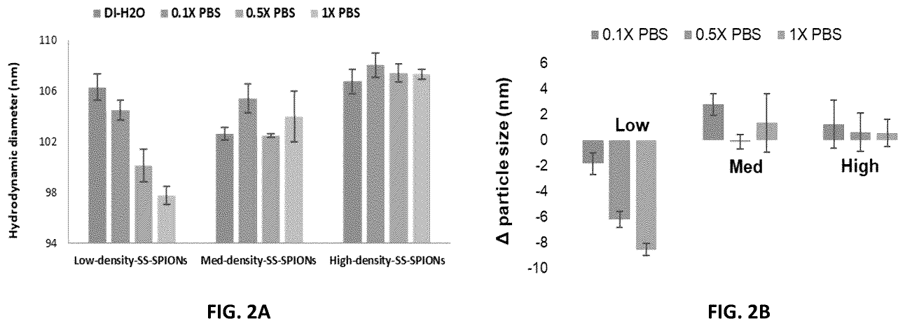

The hydrodynamic size of the polymer 104 can be a function of polymer surface density, temperature, polymer dispersity, pH, polymer molecular weight, and/or solution ionic strength. It is, therefore, possible to develop a nanoparticle construct 100 with a surface-grafted polymer 104 that has a given average size in storage solution at room temperature but which changes size upon exposure to body tissue (e.g., blood, serum, lymph fluid) at physiological temperatures. In addition to temperature change, a difference in the ionic strength of the body tissue and the storage solution can also produce a change in the size of the nanoparticle construct 100 (see FIGS. 2A-2B). Therefore, the maximum and minimum size of the nanoparticle construct 100 in different microenvironments can be determined by the molecular weight of the polymer 104 attached or grafted on the surface of the nanoparticle construct 100. The rate of size change of the nanoparticle construct 100 can be a function of the grafting density of the polymer 104 on the surface of the nanoparticle construct 100 (see FIGS. 2A-2B). The density of polymer 104 on the nanoparticle core 102 can be the threshold density required for size change. The threshold density of the polymer 104 can be about one third, about one quarter, about one fifth, or about one tenth of a theoretical maximum density of the polymer 104 on the nanoparticle core 102. In some embodiments, the storage solution can comprise an additive that prevents shrinkage in size of the nanoparticle construct 101. Dilution of the additive when the nanoparticle construct 101 is administered to a subject can result in size change.

In some embodiments, the rate of change of size of the nanoparticle construct 100 can be controlled with the addition of free polymer molecules. These free polymer molecules can maximize the size of the nanoparticle construct 100, similar to high-density surface grafting when the nanoparticle construct 100 is kept in a storage solution. However, administration of the nanoparticle construct 100 to the blood or other body tissue can produce a diluting effect, allowing the grafted polymer 104 to collapse and therefore, shrink the size of the nanoparticle construct 100.

In some embodiments, the nanoparticle construct 100 can include surface-adsorbed molecules that show a different solubility in the storage solution compared to when administered to a subject. The surface adsorbed molecules can include, but are not limited to polyacrylic acid, citric acid, hyaluronic acid, poly(acrylic-co-maleic acid), and any combination thereof. The amount of surface coating of the nanoparticle construct 100 with such surface adsorbed molecules can range from about 10% to about 20%, about 20% to about 40%, about 40% to about 60%, about 60% to about 80%, about 80% to about 100%, including any percentage and range comprised therein.

In some embodiments, the polymer 104 can include additional components or molecules that promote reversible “crosslinking” between the grafted polymers 104. Such “crosslinking” components or molecules can include compounds with charged functional groups (e.g. phosphates, sulfates, carboxylic acids, quaternary amines, etc.) that change charge upon administration or which are competitively released or cleaved by blood components. Alternatively, or additionally, in some embodiments, the polymer 104 could include compounds that cause a shift in hydrophobicity-hydrophilicity of the polymers 104 upon administration.

The polymer 104 can be susceptible to hydrolysis due to chemical, mechanical, thermal, photolytic, and/or biological processes. The hydrolysis process may produce a change in the hydration state of the polymers 104, a change in polymer 104 stiffness, and/or a change in the molecular weight of the polymer 104. Furthermore, hydrolysis can occur along the backbone of the polymer 104 or on the pendant side molecules of the polymer 104. For example, the hydrodynamic radius of poly(vinyl alcohol) (PVA) is a function of its ability to block and its molecular weight. Polyacrylamides having various degrees of hydrolysis have also shown changes in hydrodynamic size.

In another aspect, the disclosure is directed to a method of imaging comprising the steps of creating a size-changing imaging agent, administering the size-changing imaging agent to a subject, measuring a signal from the size-changing imaging agent, and removing the size-changing imaging agent from the subject after the size-changing imaging agent decreases in size. In one embodiment, the size-changing agent is the nanoparticle construct 100. In one embodiment, the size-changing imaging agent can be used for in vivo, in vitro, and/or ex vivo imaging.

In one embodiment, the method can further comprise storing the imaging agent or the nanoparticle construct 100 in a storage solution with a first temperature, a first ionic strength, and a first pH before administering the imaging agent or the nanoparticle construct 100 into a subject where the imaging agent or the nanoparticle construct 100 is exposed to a different temperature, ionic strength, and/or pH.

The shrinkage of the nanoparticle construct 100 allows for the removal of the nanoparticle construct 100 from circulation by renal clearance. In some embodiments, the diameter of the nanoparticle construct 100 can shrink from larger than about 10 nm to smaller than about 8 nm or less. In other embodiments, the diameter of the nanoparticle construct 100 can shrink from about 15 nm to about 7 nm or less. Such shrinkage in size allows for renal clearance of the nanoparticle construct 100. In some embodiments, the diameter of the nanoparticle construct 100 can shrink to about 5 nm or less. Such shrinkage allows tissue extravasation and whole-body contrast-enhanced magnetic resonance imaging. In some embodiments, renal perfusion imaging can be used for detecting the nanoparticle construct 100 after administration. Renal perfusion imaging can be used for determining split renal function as part of treatment planning of unilateral renal diseases, for assessing renal transplants, and/or for diagnosing renal cell carcinoma (RCC) by measuring tumor fractional blood volume before and after treatment. Renal perfusion imaging can be used in patients suffering from chronic kidney disease (CKD), for measuring complement GFR and assessing possible causes of CKD (e.g. renal stenosis).

In some embodiments, MRI imaging of brain lesions can be performed by using the nanoparticle construct 100. The initial size of the nanoparticle construct 100 (e.g., about 10 nm to about 30 nm) allows for the nanoparticle construct 100 to accumulate in areas with disrupted blood-brain barrier, at a faster pace compared to other large nanoparticles. The shrinkage in the size of the nanoparticle construct 100 allows for renal clearance. In some embodiments, the initial size of nanoparticle construct 100 can allow for dynamic susceptibility contrast (DSC) perfusion MRI of the central nervous system (e.g. brain lesions, stroke, Alzheimer's), either during first pass or during the steady state MRI phase, with reduced leakage through the disrupted blood brain barrier compared to gadolinium based contrast agents (GBCAs). The shrinkage in the size of the nanoparticle construct 100 in such applications can allow for delayed renal clearance.

In some embodiments, imaging of tumor angiogenesis can be performed by using the nanoparticle construct 100. Vessel permeability and tumor perfusion (e.g. assess tumor treatment, tumor heterogeneity) can be evaluated by measuring microvessel parameters. For example, microvessel density can be imaged as a prognostic measure in different cancer types as blood vessel density can vary depending on the cancer type. Imaging can be done for pre-procedural planning for transarterial embolization of vascular neoplasms, such as for hepatocellular carcinoma.

In another aspect, the disclosure is directed to a method of fabricating a size-changing imaging agent (e.g., the nanoparticle construct 100), comprising the steps of synthesizing a nanoparticle core by co-precipitation, stabilizing the nanoparticle core 102 by using a surface coating, and grafting a polymer 104 to the nanoparticle core 102.

### Example 1: Grafting Density of Polymers on the Nanoparticle Construct

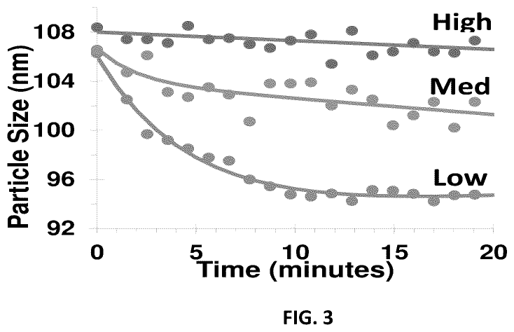

In an illustrative example, as shown in FIGS. 2A, 2B, and 3, the hydrodynamic size of the nanoparticle construct comprising PEG polymers was altered as a function of both polymer density and solution ionic strength. The maximum size obtained for the nanoparticle construct was the same regardless of the polymer grafting density. The nanoparticle constructs with low-density PEG, however, showed a decrease in size as the solution ionic strength was increased to that of human blood (1× PBS). A maximum size change of about 9 nm was observed while the nanoparticle construct with medium and high-density PEG showed reduced sensitivity to ionic strength. The rate of size change of the nanoparticle construct was also dependent on PEG density (FIG. 3).

### Example 2: Evaluation of Hydrodynamic Size-Change with Varying PEG Molecular Weight (MW) and Surface Coverage

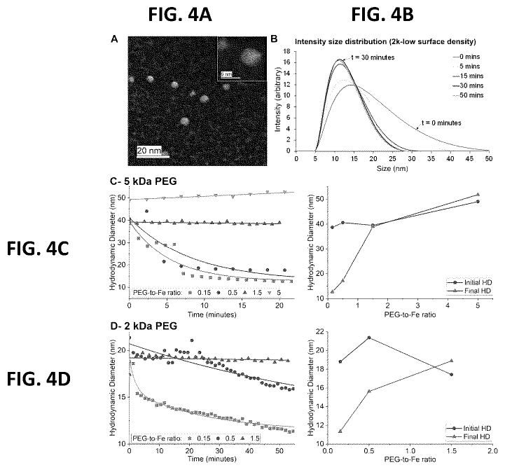

PEGylation of PAA-SPIONs (SPIONS coated with PAA) (FIG. 4A) was conducted under varying reaction conditions, including varying PEG molecular weight, concentrations, buffers, reaction time, and reaction temperature. For size-change experiments, nanoparticle constructs initially suspended in deionized water were transferred to saline, which mimics the ionic strength in blood. Measurement of the hydrodynamic particle diameter by dynamic light scattering (DLS) generated size distribution curves such as those shown in FIG. 4B. Nanoparticle constructs that shrink over time display a narrowing of the size distribution curves and an overall shift to lower average hydrodynamic size. It was shown that PEG molecular weight and coating density had a significant effect on nanoparticle construct size and size change.

PAA-SPIONs were PEGylated with 5 kDa PEG at varying PEG-to-Fe ratios ranging from 0.15 to 5. Looking only at initial sizes given by t=0 min data points in FIG. 4C, PEG:Fe ratios up to 1.5 yielded nanoparticle constructs with initial hydrodynamic sizes of approximately 40 nm and it was increased to 49 nm for PEG:Fe ratio of 5. This increase in size is expected as polymers packed at a high density would take a more extended confirmation. Furthermore, it was observed that nanoparticle constructs modified with PEG:Fe ratio of 1.5 or greater did not exhibit any size change with time. This is again likely due to a high packing of PEG, preventing collapse of the polymer to reduce size. Additionally, with PEG:Fe of 0.5 and lower, nanoparticle construct size decreased significantly by 22+ nm, from initial to final, over 20 mins, while no change is observed with higher ratios (FIG. 4C right panel). From this study, a critical PEG:Fe ratio under which size-changing nanoparticle constructs can be obtained was determined. Nanoparticle constructs modified with 5 kDa PEG at a PEG:Fe ratio 0.15 (5k-SCNP) was one formulation selected for further studies.

A follow-up study was then conducted using 2 kDa PEG, as shown in FIG. 4D. Again, the PEG density had a significant effect on the extent and kinetics of size shrinkage. Nanoparticle constructs with high PEG coverage did not show size shrinkage over a period of about 52 minutes. The lowest density nanoparticle constructs, however, showed excellent size shrinkage with a size decrease of about 7 nm. The kinetics of change showed two phase kinetics, initially rapid and then slowing, and approached the hydrodynamic size of the base unmodified nanoparticle constructs. This was selected to be the second potential formulation (2k-SCNP) for further studies.

### Example 3: Surface Coating of the Nanoparticle Construct

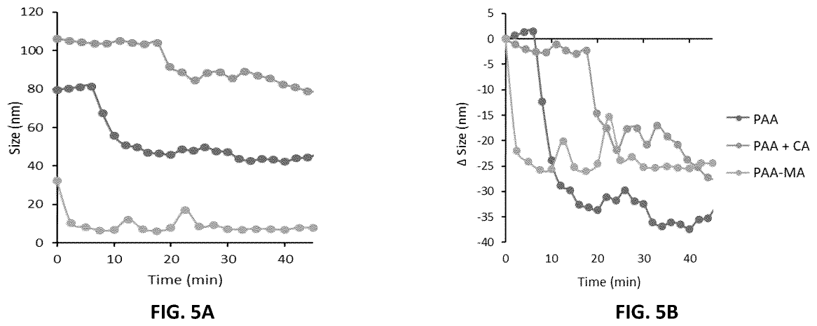

Nanoparticle constructs synthesized using co-precipitation method can be stabilized with different surface coatings including 100% polyacrylic acid (PAA), polyacrylic acid and citric acid (PAA+CA), and polyacrylic acid and poly(acrylic-co-maleic acid) (PAA-MA) before being modified with polyethylene glycol (PEG) (e.g., about 5 kDa molecular weight). Size shrinkage was observed with dynamic light scattering (DLS) when the nanoparticle constructs were transitioned from water (time 0) (see FIG. 5A) to 1× PBS with a higher ionic strength than water (see FIG. 5B).

### Example 4: Varying the Density of Polymers

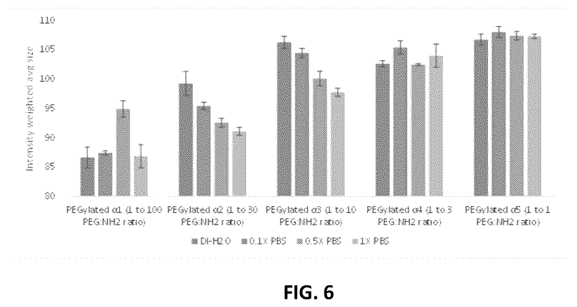

Low, medium, and high polymer density nanoparticle constructs were produced by controlling the ratio of amine reactive N-hydroxylsuccinimide (NHS) functionalized polyethylene glycol (PEG-NHS) to the number of amine (NH₂) sites on the nanoparticle construct surface as shown in FIG. 6. For the low, medium, and high samples the PEG:NH₂ ratios used were about 1:10, 1:3, and 1:1, respectively. The high density was close to the maximum density. Nanoparticle constructs with even lower PEG density (1:100 and 1:30 ratio of PEG:NH₂) were also synthesized.

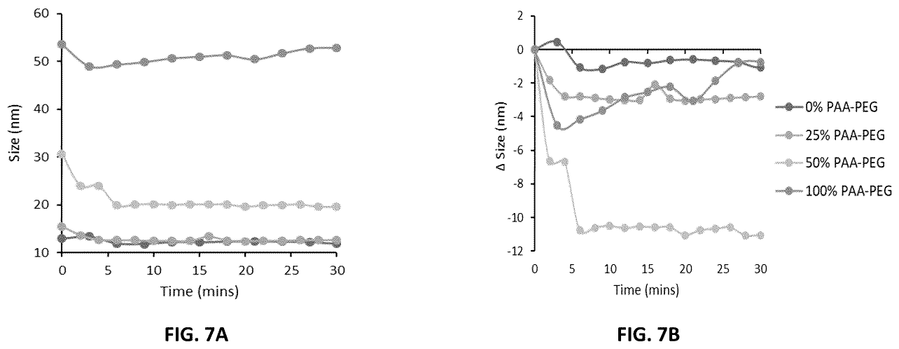

Nanoparticle constructs with superparamagnetic iron oxide nanoparticle core (SPIONs) were synthesized, and stabilized with polymeric surface coatings at varying molar ratios of polyacrylic acid (PAA) and polyacrylic acid-poly(ethylene glycol) block copolymer (PAA-PEG) as shown in Table #1 and FIGS. 7A-7B. 25% PAA-PEG comprised a low PEG density with a mushroom conformation. In such nanoparticle constructs, a negligible shrinkage was observed with increasing ionic strength. 50% PAA-PEG comprised a medium PEG density. In such nanoparticle constructs, the highest shrinkage was observed with increasing ionic strength. 100% PAA-PEG comprised a maximum surface PEG density with a brush conformation. In such nanoparticle constructs, a negligible shrinkage was observed with increasing ionic strength.

### Example 5: Inclusion of a “Free” Polymer that is Diluted Upon Administration

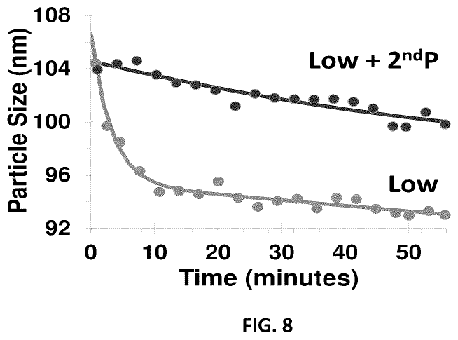

In an illustrative example, as shown in FIG. 8, low-density PEG polymers resulted in a rapid decrease in the size of the nanoparticle construct within about 10 minutes after introduction to 1× PBS. The inclusion of a second free PEG, however, significantly reduced the rate of size change. For example, even one hour after introduction to 1× PBS, the reduction in size was not the same as what was observed without the free PEG in less than a minute.

TABLE &num;1

| | | | Initial | Final size | Δ Size |
|---|---|---|---|---|---|
| | Surface Coating | | size | (nm) in | change (nm) |
| | PAA- | | (nm) in | 1X size | with increased |
| Name | PEG | PAA | water | PBS | ionic strength |
| 0% PAA-PEG | 0% | 100% | 12.92 | 12.71 | −0.21 |
| 25% PAA-PEG | 25% | 75% | 15.44 | 12.88 | −2.56 |
| 50% PAA-PEG | 50% | 50% | 30.63 | 19.47 | −11.16 |
| 100% PAA- | 100% | 0% | 53.50 | 51.77 | −1.73 |
| PEG | | | | | |

### Example 6: Incorporating β-Cyclodextrin in the Nanoparticle Construct

The system can also take advantage of polymer systems comprised of multiple components, the interaction of which varies with changes in the microenvironment. For example, polycarbohydrates such as alginate and chitosan, which show pH dependent charge behavior, can be utilized together. Additionally, the polymer can consist of a composite molecule such as polyrotaxane, which is comprised of PEG molecules threaded through multiple cyclodextrins. With cyclodextrin, the PEG can have a stiffer and more elongated configuration. The size and stability properties of the nanoparticle construct can change if upon administration the cyclodextrin were to become unthreaded. The rate of cyclodextrin loss can be controlled by the type of cyclodextrin (i.e. size) and the size of the capping group on the PEG.

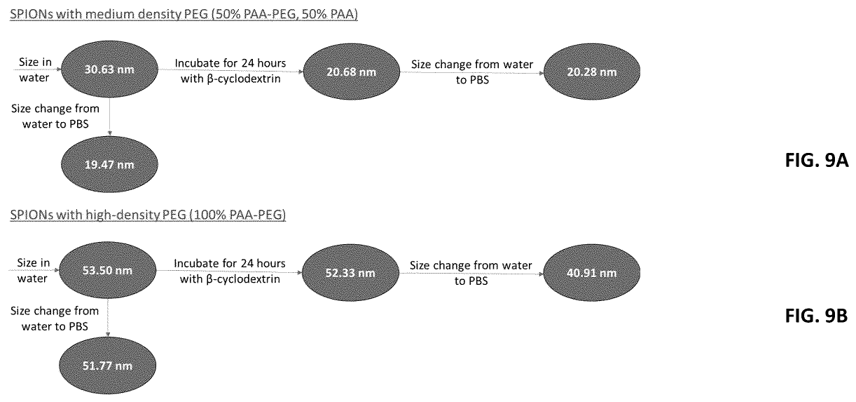

As shown in FIGS. 9A-9B, the incorporation of β-Cyclodextrin, a cyclic oligosaccharide with a hydrophilic exterior and a hydrophobic cavity, has an effect on the size shrinkage of the nanoparticle constructs (e.g., SPIONs) with a medium density of PEG. The nanoparticle constructs experienced a similar level of size shrinkage when incubated with β-Cyclodextrin for 24 hours in water as when they were transferred from water to 1× PBS. The combined effect of both conditions had no additional effect on size shrinkage. Further, for nanoparticle constructs with a high density of PEG, significant size shrinkage was only observed when the nanoparticle constructs were first incubated with β-Cyclodextrin for 24 hours and then transitioned from water to 1× PBS.

### Example 7: Hemocompatibility of Nanoparticle Construct

Due to their abundance and size, red blood cells (RBCs) contribute to thrombosis and hemostasis primarily through their rheological effects (platelet margination, aggregation, and deformability of RBCs), but also through biochemical interactions with platelets and endothelial cells. Additionally, hemolysis, i.e. damage of RBCs and subsequent release of toxic heme, hemoglobin, and procoagulant microparticles into blood, can cause intravascular coagulation. The FDA's guidance to industry recommends that intravenously injected agents be tested early for their acute hemolytic property. Formulations with hemolysis values <10% are considered nonhemolytic, while those over 25% are at risk for causing hemolysis.

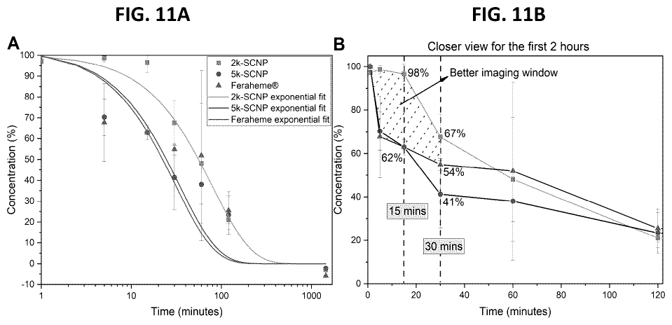

The hemocompatibility of 2k-SCNP, 5k-SCNP, and Feraheme® with RBCs was tested by a hemolysis assay as previously described. Briefly, nanoparticle constructs at different concentrations (ranging from 12.5 to 100 μg Fe/mL) in 1× Phosphate-Buffered Saline (PBS) were incubated with RBCs from Sprague-Dawley (SD) rats at 37° C. for 2 hours. This time point was chosen as the majority of the formulation is cleared from circulation at that time point (see PK results in FIG. 11B). At the end of the incubation period, the samples were centrifuged at 1000×g for 5 minutes, the supernatant collected, and its optical density (OD) measured at 580 nm. Water and PBS were used to generate the positive (100% hemolysis due to hypotonic effect of water) and negative (0% hemolysis) controls, respectively. Hemolysis ratio % was calculated as

$$\frac{( O {D}_{N P} - O {D}_{neg . control} )}{( O {D}_{pos . control} - O {D}_{neg . control} )} \times 100.$$

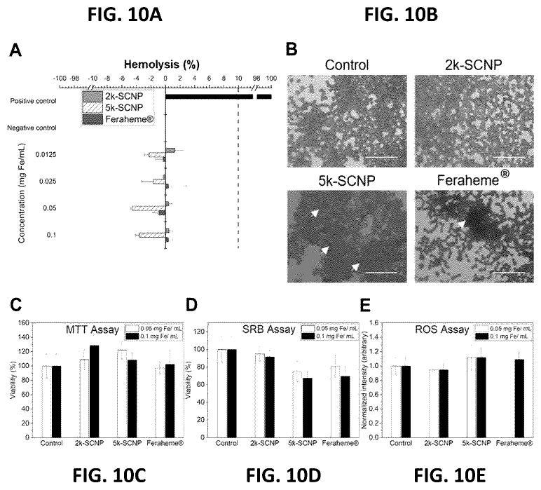

Negligible hemolysis levels (<1%) were observed for both formulations, and for Feraheme®, at 4 different concentrations (FIG. 10A), all showing data significantly under the 10% hemolysis acceptance limit for biopharmaceuticals.

Upon administration, intravenously injected foreign particles come into contact with plasma proteins that adhere onto their surface, triggering rapid uptake by macrophages of the mononuclear phagocytic system (MPS) and removal from circulation. Surface coatings are typically included in nanoparticle formulations to slow down this process. To further evaluate nanoparticle construct stability in blood circulation and mimic the MPS clearance process, 2k-SCNP, 5k-SCNP and Feraheme® (0.1 mg/mL) were incubated with Raw 264.7 macrophage cells for 2 hours (same as hemolysis test), and Prussian blue staining was used to qualitatively measure cellular SPION uptake. High uptake would signal potentially rapid removal of nanoparticles from circulation and clearance into the spleen and liver, rather than through renal filtration, while also hamper MRA performance due to reduced blood concentration and increased background noise surrounding organs of elimination.

2k-SCNP demonstrated markedly reduced macrophage uptake compared to 5k-SCNP (moderate uptake) and Feraheme® (high uptake), as shown in FIG. 10B. Cells were stained with safranin (cellular membrane stain) and Prussian blue (iron stain). White arrows show blue iron spots. Among “normal” cells, macrophages have one of the highest rates of nanoparticle construct uptake; uptake of 2k-SCNP (and to a lesser degree 5k-SCNP) is therefore expected to be negligible in vivo. However, Feraheme's mechanism of action in clinical pharmacology is to enter the MPS macrophages of the spleen, liver, and bone marrow. It was shown that the nanoparticle construct formulations do not adversely interact with blood components (<10% hemolysis).

Cytotoxicity studies were conducted on Raw 264.7 macrophage cells with two concentrations of SPIONs, using the 3-(4,5-dimethylthiazol-2-yl)-2,5-diphenyltetrazolium bromide (MTT) and sulforhodamine B (SRB) assays, which measure mitochondrial activity and cellular protein content, respectively. Results from the MTT assay (FIG. 10C) showed that tested formulations had no negative effect on cell viability. Meanwhile, results from the SRB assay (FIG. 10D) showed 2k-SCNP formulation having no negative effect on cell viability compared to control, while also meeting the preset criteria of >90% cell viability. On the other hand, 5k-SCNP at 0.05 mg/mL (p<0.01) and 0.1 mg/mL (p<0.01), and Feraheme® at 0.05 mg/mL (p<0.05) and 0.1 mg/mL (p<0.01) showed significant effect on cell viability compared to control, with cell viability <90%.

A cellular ROS assay was conducted to study the effect of the nanoparticle construct formulations and Feraheme® at two different concentrations on ROS generation in Raw 264.7 macrophage cells. The assay uses 2′,7′-dichlorofluorescin diacetate (DCFDA) to quantify ROS in live cells samples. None of the samples exhibited significant increase in ROS production vs. control (FIG. 10E). However, cells treated with 5k-SCNP and Feraheme® at 0.1 mg/mL produced significantly more ROS compared to cells treated with 2k-SCNP at the same concentration (p<0.05).

### Example 8: Pharmacokinetics (PK), Biodistribution, and Toxicity of Size Changing Nanoparticle (SCNP) Constructs in an In Vivo Rat Model

2k-SCNP, 5k-SCNP and Feraheme® were injected (0.1 mmol Fe/kg), similar to molar concentrations of gadolinium-based contrast agents (GBCAs) administered clinically into the jugular vein of catheterized SD rats, and blood samples taken at 1, 5, 15, 30 mins, 2, and 24 hr, and iron oxide nanoparticle concentrations quantified. Nanoparticle plasma concentration vs. time (FIG. 11A) was fitted using a PK first order elimination model, C=Cₒe^{-kt}, where C is the concentration at time, “t”, Cₒ is the initial concentration in plasma, and k is the elimination rate constant. ³²Plasma half-life (t_{1/2}), equal to In (2)/k, is the time needed for the concentration in the plasma to reach half of the initial concentration. The half-lives (t_{1/2}) were 21.26 min, 23.56 min, and 55.32 min for Feraheme®, 5k-SCNP, and 2k-SCNP, respectively.

PK results from animals injected with 2k-SCNP showed plasma concentration remained constant in blood for 15 minutes post-injection (˜98% of maximum plasma concentration at 15 min; consistent in all tested animals), and then decreased sharply afterwards (FIG. 11B). On the other hand, animals injected with 5k-SCNP and Feraheme® experienced a sudden and rapid decrease in plasma concentrations (˜62% concentration at 15 minutes for both formulations). This 15-minute time window with near maximum plasma concentration with 2k-SCNP formulation provides ample time for high-resolution contrast enhanced-MRA (CE-MRA) in the steady-state regime (confirmed through interviews with radiologists).

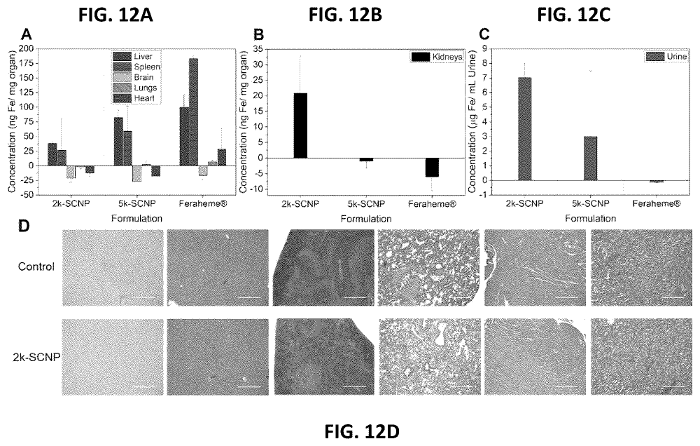

Biodistribution analysis, conducted in tandem with the PK study, quantified concentrations of SCNP and Feraheme® in major organs. Animals were euthanized at 24 hours post-injection and tissues (liver, spleen, kidneys, heart, lungs, and brain) were collected for determination of SPION concentrations and for toxicity analysis. Significant accumulation of Feraheme® was observed in the liver and spleen (FIG. 12A), and no accumulation observed in the kidneys (FIG. 12B). This was an expected result as Feraheme® is taken up by MPS macrophages of the liver, spleen, and bone marrow. The ultrasmall SCNPs, on the other hand, were expected to be cleared primarily through renal filtration.

However, significant accumulation of 5k-SCNP was observed in the liver and spleen, but none in the kidneys. It is hypothesized that the surface density of 5 kDa PEG was not sufficient to shield the nanoparticle constructs from rapid macrophage uptake and removal from circulation into the liver and spleen. Finally, less than one third accumulation of 2k-SCNP was observed in the liver compared to Feraheme®, and less than one sixth accumulation in the spleen compared to Feraheme® (FIG. 12A) and significantly more accumulation of 2k-SCNP in the rats' kidneys than 5k-SCNP and Feraheme® (FIG. 12B). There was no detectable accumulation of 2k-SCNP and 5k-SCNP in the lungs, heart, and brain. Significant accumulation of Feraheme® was observed in the heart, but not in the lungs and the brain.

Urine analysis confirmed renal clearance of 2k-SCNP while Feraheme® was undetectable in urine samples at 24 hours (FIG. 12C). Additionally, in a later MRA study, urine was collected at 1 hour for animals injected with 2k-SCNP and Feraheme® and results indicate that concentration of 2k-SCNP at 1 hour was twice as much as at 24 hours. Feraheme® was undetectable at 1 hr and 24 hrs. Therefore, it has been illustrated that 2k-SCNP undergoes renal clearance.

Changes in tissue morphology were determined with statistically significant changes from control group indicating toxicity. Organs (spleen, liver, kidneys, heart, brain and lungs) were collected at 24 hr post-injection, and ˜ half of each organ was fixed in 10% neutral buffered formalin. Tissues were then embedded in paraffin blocks, frozen at 4° C., sectioned, and stained with hematoxylin and eosin (H&E) for pathological examination to determine possible SPION-induced tissue damage. Histological images show insignificant pathological changes (no apparent tissue damage, lesions or inflammation) in animals treated with 2k-SCNP, 5k-SCNP, or Feraheme® (FIG. 12D) showing images for control and 2k-SCNP). Additionally, whole blood analysis showed no significant differences in hematocrit values (percentage of red blood cells in blood) between rats injected with our formulations and control rats.

### Example 9: MRA Performance in a Rat Model

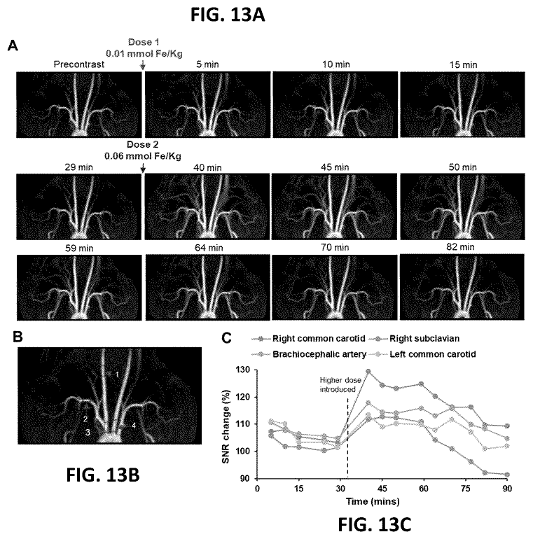

A preliminary CE-MRA study with Sprague-Dawley (SD) rats was conducted following injection of 2k-SCNP into the jugular vein of cannulated SD rats. Pre-contrast and post-contrast T 1-weighted MRA images were taken of the neck region, highlighting the aortic arch, coronary, vertebral, brachiocephalic, and subclavian vessels (among others). In this study, 0.01 mmol/kg was initially injected (10% dose of GBCAs administered clinically), and multiple MRA scans taken afterwards, as shown in FIG. 13A. An additional 0.06 mmol/kg of 2k-SCNP was injected at t=34 min, and MRA scans taken. Regions of interest (ROI) spanning multiple blood vessels (right common carotid, right subclavian, brachiocephalic, and left common carotid arteries) were chosen for analysis (FIG. 13B).

Signal-to-noise ratio (SNR) is defined as ratio of mean signal intensity in ROI to standard deviation of signal intensity in air. SNR change (%) is defined as SNR at time, “t”, relative to SNR at pre-contrast level. Values over 100% indicate enhanced SNR in ROI relative to pre-contrast. Post-contrast imaging with SCNP, even at low dose, had SNR change >100% for all four ROIs during the entire duration (first 29 minutes) (FIG. 13C), which establishes its superiority over non-contrast MRA. Meanwhile, injecting an additional 0.06 mmol/kg of 2k-SCNP resulted in much higher SNR change in all ROIs, and signal remained relatively constant for 25 minutes after (from t=34 min to t=59 min), before signal started to drop. This validates delayed clearance of 2k-SCNP.

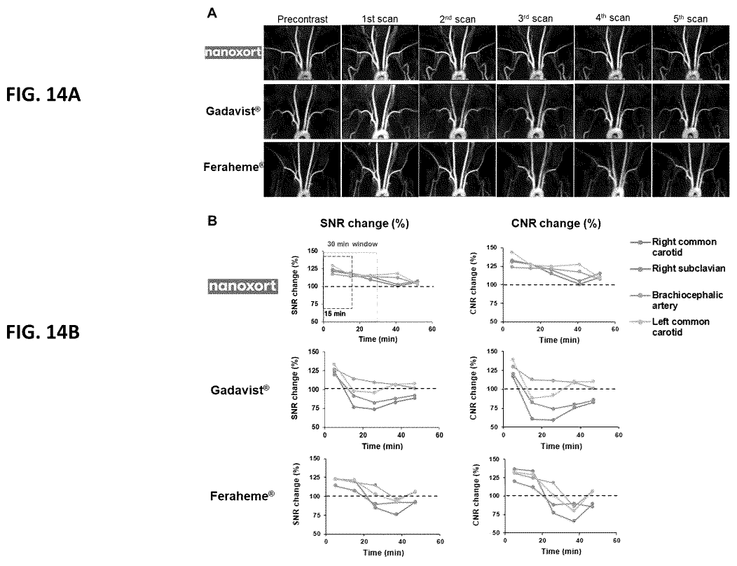

In a subsequent study, MRA performance of 2k-SCNP was directly compared to the extracellular GBCA Gadavist® and the iron-based agent Feraheme® in SD rats. It is hypothesized that the extended vascular imaging window provided by the iron-based nanoparticle construct will yield improved MRA imaging of the vascular system versus GBCAs, especially at later time points. Pre-contrast and post-contrast images (n=5) were acquired (FIG. 14A).

In addition to calculating SNR change (%), ROIs near each vessel of interest in less vascularized/perfused surrounding tissues were used to calculate contrast to noise ratio (CNR, equal to difference in signal intensity between vessel and less vascularized surrounding region, divided by standard deviation of signal intensity in air). CNR change (%) compares CNR at time, “t”, relative to CNR at pre-contrast level.

While SNR change (%) and CNR change (%) values were similar for all three agents during the first scan (t=5 min), only with 2k-SCNP did the values remain >100% for all 4 ROIs during the entire scan time (FIG. 14B). The signal remained within 15% of maximal levels for the first 26 minutes post-injection; establishing 2k-SCNP as a blood pool contrast agent (BPCA). With Gadavist®, SNR change (%) and CNR change (%) values dropped below 100% during the second scan (t=15 min) for three of the four tested ROIs, and decreased even further during the third scan (t=26 min). With Feraheme®, CNR change (%) values dropped below 100% for 2 of the 4 ROIs during the third scan (t=26 min), and for all four ROIs during the fourth scan (t=37 min).

What is claimed is:

1. An agent comprising:
a nanoparticle core, and
a polymer attached to a surface of the nanoparticle core,
wherein the agent is configured to change in size upon exposure to a body tissue of a subject.

2. The agent of claim 1, wherein the polymer is a first polymer, and wherein the imaging agent further comprises a second polymer.

3. The agent of claim 1, wherein the polymer is PEG.

4. The agent of claim 1, wherein the agent is stored in a storage solution with a first temperature before being administered into the body tissue with a second temperature, and wherein the polymer is configured to change in size after administration if the first temperature is different from the second temperature.

5. The agent of claim 1, wherein the agent is stored in a storage solution with a first ionic strength before being administered into the body tissue with a second ionic strength, and wherein the polymer is configured to change in size after administration if the first ionic strength is different from the second ionic strength.

6. The agent of claim 1, wherein the agent is stored in a storage solution with a first pH before being administered into the body tissue with a second pH, and wherein the polymer is configured to change in size after administration if the first pH is different from the second pH.

7. The agent of claim 1, wherein the polymer is configured to change in size at a rate determined by pH, ionic strength, or temperature of the body tissue of the subject.

8. The agent of claim 1, wherein the polymer is configured to undergo hydrolysis in the body tissue of the subject.

9. The agent of claim 1, wherein the change in size of the agent is dependent on a threshold density of the polymer.

10. The agent of claim 1, wherein the body tissue is blood.

11. The agent of claim 1, wherein the body tissue is a tumor microenvironment.

12. The agent of claim 1, wherein the polymer is configured to target a biomarker in the subject.

13. The agent of claim 1, wherein a threshold density of the polymer is tethered to the nanoparticle core.

14. The agent of claim 13, wherein the threshold density of the polymer is one half of a theoretical maximum density of the polymer on the nanoparticle core.

15. The agent of claim 13, wherein the threshold density of the polymer is one third, one quarter, one fifth, or one tenth of a theoretical maximum density of the polymer on the nanoparticle core.

16. The agent of claim 1, wherein the nanoparticle core comprises magnetic material.

17. The agent of claim 1, wherein the agent further comprises a cyclic oligosaccharide with hydrophilic exterior and hydrophobic cavity.

18. The agent of claim 1, wherein agent is configured to change in size from above about 10 nm to below about 8 nm upon exposure to the body tissue.

19. The agent of claim 1, wherein agent is an imaging agent.

20. A method of imaging comprising:
creating a size-changing imaging agent,
administering the size-changing imaging agent into a subject,
measuring a signal from the size-changing imaging agent, and
removing the size-changing imaging agent from the subject after the size-changing imaging agent changes in size.
